(19) United States
(12) Patent Application Publication
(10) Pub. No.: US 2010/0145147 A1
(43) Pub. Date: Jun. 10, 2010

(54) MAGNETIC DEVICE FOR GUIDING CATHETER AND METHOD OF USE THEREFOR

(75) Inventors: Michael Raymond Pinsky, Pittsburgh, PA (US); Juan Bernardo Ochoa Gautier, Pittsburgh, PA (US); Josef Winkler, Wayland, MA (US); Gary Wakeford, Canfield, OH (US); Sabry Gabriel, Lizella, GA (US); Paul Frankhouser, Miami Beach, FL (US); Aaron Sandoski, Lincoln, MA (US)

Correspondence Address: Pabst Patent Group LLP, 1545 PEACHTREE STREET NE, SUITE 320, ATLANTA, GA, 30309, US

(73) Assignees: Syncro Medical Innovations, Inc.; University of Pittsburgh - Of the Commonwealth System of Higher Education

(21) Appl. No.: 12/553,019

(22) Filed: Sep. 2, 2009

Related U.S. Application Data
(60) Provisional application No. 61/093,628, filed on Sep. 2, 2008.

(51) Int. Cl. A61B 1/00

(52) U.S. Cl. 600/114

(57) ABSTRACT

One or more external magnets that are suitable for placement on the back or front of a patient's neck and methods for using the external magnet(s) to guide a feeding tube containing one or more internal magnets through the esophagus are described herein. The external magnet is applied to the back or the front of the patient's neck and the feeding tube is inserted into the patient's nose and advanced within the patient's body. The magnetic field of the external magnet combines with the magnetic field of the internal magnet to produce a sufficient magnetic force to pull or push the feeding tube apparatus against the posterior wall of the esophagus to prevent placement of the stylet in the patient's trachea or windpipe to prevent insertion into the patient's lungs.

### CROSS-REFERENCE TO RELATED APPLICATION

This application claims priority to U.S. Provisional Application Ser. No. 61/093,628, entitled “Magnetic Device for Guiding Catheter”, filed Sep. 2, 2008. The disclosure of this application is incorporated herein by reference.

### FIELD OF THE INVENTION

The present invention relates to methods and devices for placement of feeding tubes.

### BACKGROUND OF THE INVENTION

A reported problem with all feeding tubes is misplacement of the tube in the airway. Inadvertent insertion of nasogastric tubes into the trachea and distal airways is reported to range from 0.3% to 15% of insertions. (see Thomas et al., Journal of the American College of Nutrition, 17(2):195-197 (1998)). Both airway penetration and introduction of various chemicals into the lung and pleural spaces may occur prior to recognition of tube misplacement and can be fatal. Currently, nursing staff checks the location of the feeding tube using x-ray during the insertion process. This requires stopping the procedure to perform the x-ray and thereby increases the time, cost and inefficiency involved in this procedure.

A variety of feeding tubes have been developed to improve the ease of insertion and the ability of medical practitioners to ensure proper placement of the feeding tube in the desired location. For example, U.S. Pat. No. 5,431,640 to Gabriel discloses a method and apparatus for intubation of a patient using a magnetic field produced between an external magnet and a magnet at the distal end of a catheter to maneuver the catheter to the distal duodenum of a patient.

U.S. Pat. No. 6,126,647 to Posey, et al., discloses a catheter guided by an external magnet, which contains a sensor that indicates whether the distal end of the catheter is being properly advanced into the patient's duodenum. The catheter contains a magnet that is permanently affixed in the distal portion of the catheter.

Although each of the above-described catheters provide improvements over other feeding tubes that are available, none of the feeding tubes is designed to ensure that the catheter is not accidentally placed in the trachea without the need for x-rays to confirm the location of the feeding tube.

There is a need for improved methods for directing a feeding tube through a patient's esophagus.

Therefore, it is an object of the invention to provide improved methods for directly a feeding tube through a patient's esophagus.

It is a further object to provide devices to facilitate directing a feeding tube through a patient's esophagus.

### SUMMARY OF THE INVENTION

One or more external magnets that are suitable for placement on the back or the front of a patient's neck and methods for using the external magnet(s) to guide a feeding tube through the esophagus are described herein. The external magnet(s) are designed to be placed on the back or the front of the patient's neck and may be used with a magnetic feeding tube. The magnetic feeding tube may contain one or more magnet(s) or magnetically attractive material(s) (the “internal magnets”) that is attached to the feeding tube or one that is removable from the feeding tube. In one preferred embodiment, the feeding tube contains one or more magnet(s) or magnetically attractive material(s) (also referred to as the “internal magnets”) that are removable from the catheter. The external magnet is applied to the back or the front of the patient's neck and the feeding tube is inserted into the patient's nose and advanced within the patient's body. The magnetic field of the external magnet combines with the magnetic field of the internal magnet to produce a sufficient magnetic force to pull or push the feeding tube apparatus against the posterior wall of the esophagus to prevent placement of the stylet in the patient's trachea or windpipe to prevent insertion into the patient's lungs. Once the catheter is advanced beyond the patient's neck, the external magnet may be removed from the patient's neck. In some embodiments, the same or a second, different external magnet is used to advance the feeding tube apparatus to the desired location for administering food and/or medicine to the patient. In the embodiments utilizing a one or more magnet(s) or magnetically attractive material(s) that are removable from the catheter, the one or more magnet(s) or magnetically attractive material(s) (e.g. a magnet stack) may be removed when the feeding tube reaches the desired location. This allows for the catheter to remain in place while the patient undergoes diagnostic testing, such as magnetic resonance imaging.

### BRIEF DESCRIPTION OF THE DRAWINGS

FIGS. 1A-D are representative illustrations of suitable shapes for the one or more external magnets.

FIGS. 2A and 2B are representative images of suitable neck braces and collars, which can contain the external magnet.

FIGS. 3A-C illustrate three representative configurations for the hand held external magnet.

FIG. 4A is a schematic of a feeding tube apparatus with an indicator. FIG. 4B is a schematic of the feeding tube apparatus without an indicator.

FIG. 5 illustrates a cross-sectional view of a catheter formed of a reinforced material.

FIG. 6A illustrates the cross-section of the distal end of a stylet, which contains a magnet stack. FIG. 6B illustrates the cross-section of the distal end of a feeding tube apparatus, which contains a reed switch assembly.

FIG. 7 illustrates a cross-section of the reed switch assembly.

### DETAILED DESCRIPTION OF THE INVENTION

### I. External Magnet for Guiding Feeding Tube

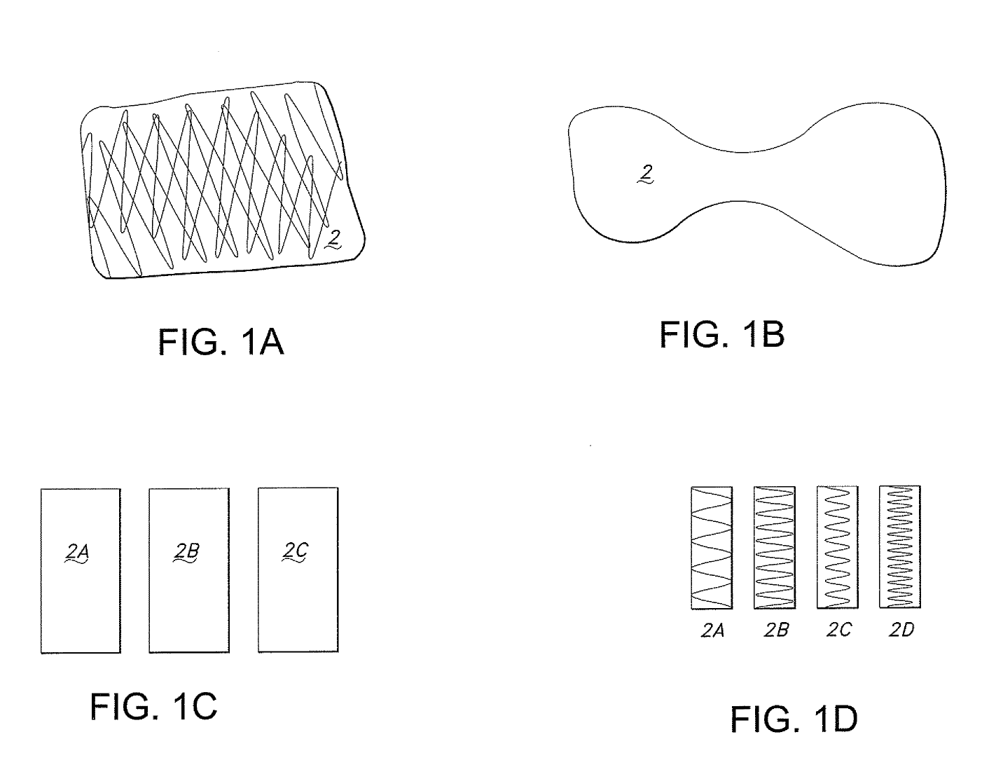

One or more external magnets (2) (see e.g. FIGS. 1A-D) that are suitable for placement on a patient's neck may be used to guide a feeding tube through the esophagus by directing the distal tip of the feeding tube to the posterior wall of the esophagus.

A. Neck Brace or Collar

The one or more external magnets (2) may be located within a material that is designed to be placed on the neck, such as a neck brace or cervical collar (4). Any standard neck brace or cervical collar can be modified to contain one or more magnets. Optionally, the neck brace or cervical collar contains one or more pockets designed to contain one or more magnets. Optionally, the external magnet(s) are insertable into and removable from the neck brace or collar.

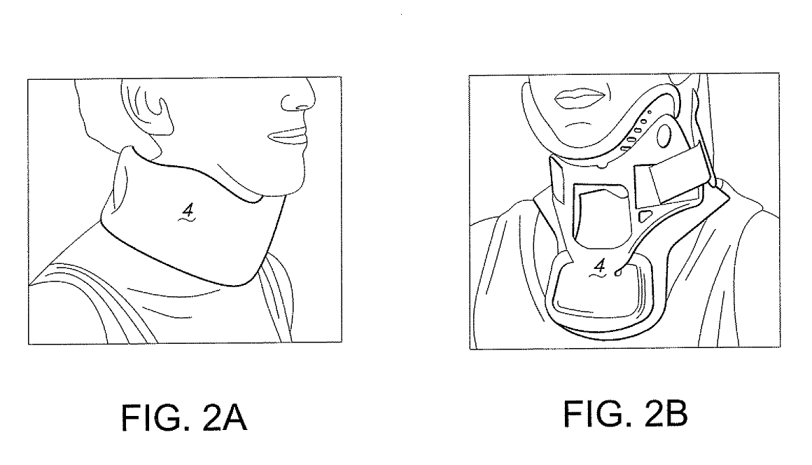

In one embodiment, the neck brace or collar is formed from a soft pliable material (see e.g. FIG. 2A). In another embodiment, the neck brace or collar is formed from a rigid plastic material (see e.g. FIG. 2B).

B. Adhesive

In another embodiment, one side of the external magnet may contain an adhesive material that is suitable for temporarily adhering to the patient's skin when the magnet is placed on the front or back of the patient's neck. In a preferred embodiment, the adhesive material is covered with a non-stick removable covering, such as a coated paper. The removable covering protects the adhesive surface prior to placement on the patient's skin. When the external magnet (2) is ready to be used, the non-stick covering is removed and the magnet is placed on the back or the front of the patient's neck, depending on the relative polarities of the internal and external magnets.

C. Hand Held External Magnet

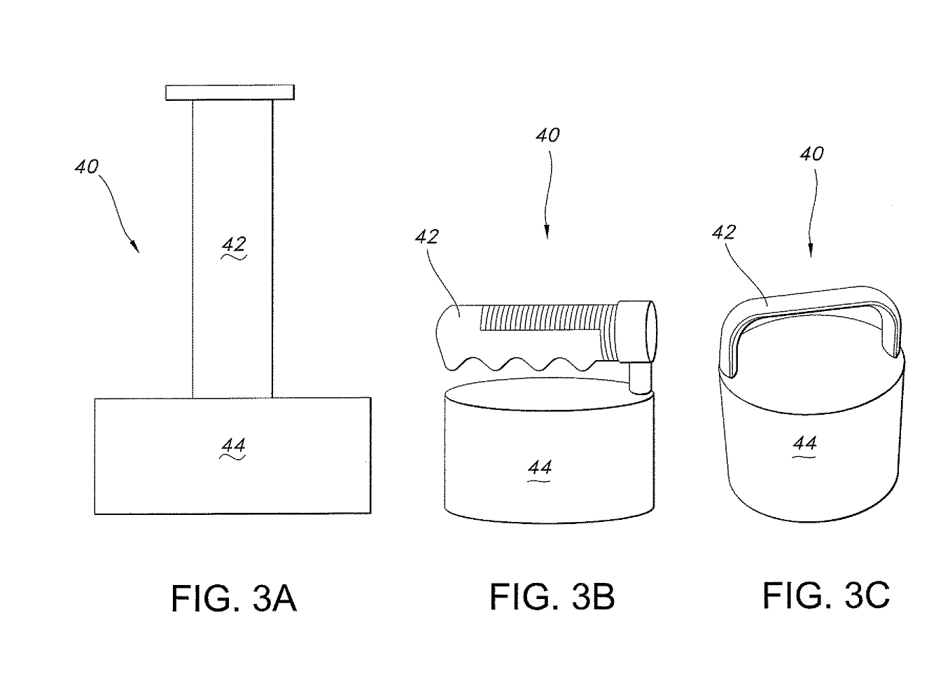

In an alternative embodiment, the magnet may be a hand-held magnet that is placed on, either behind or in front of, the patient's neck, such as by laying it against or inside a pillow upon which the patient places the back of his/her neck, or by holding it against the back of the patient's neck. The magnet may have any suitable shape and size to provide a magnetic field that, when it combines with the magnetic field of the internal magnet, produces a sufficient magnetic force to pull or push the feeding tube apparatus against the posterior wall of the esophagus to prevent placement of the stylet in the patient's trachea or windpipe. FIGS. 3A-C depict three representative designs for the external magnet. The external magnet may include a handle (42) attached to a base magnet (44). The handle may be affixed perpendicularly to the base magnet (44), as shown in FIG. 3A. Alternatively, the handle may be attached to the base magnet (44) so that it is parallel with the base magnet (44), as shown in FIGS. 3B and 3C.

In one embodiment, a pillow contains a compartment for inserting the handheld magnet. The compartment has a size and shape that generally corresponds with the size and shape of the handheld magnet

i. Dimensions and Weight

The base magnet may have a wide range of dimensions and shapes. In a preferred embodiment, the base magnet contains a surface distal to the handle that is flat. This surface is designed to be placed in contact with the patient's neck. In one preferred embodiment, the base magnet is in the shape of a cylinder.

Typical diameters for the base magnet range from about 1 inch to about 6 inches, preferably from about 3 inches to about 5 inches, more preferably about 4 inches. Typical heights for the base magnet range from about 0.25 inches to about 3 inches, preferably from about 1.5 inches to about 2.5 inches, more preferably 1.8 inches.

Preferred base magnets are as light as possible while providing the required magnetic field to combine with the magnetic field of the internal magnet to produce a sufficient magnetic force to pull or push the feeding tube apparatus against the posterior wall of the esophagus to prevent placement of the stylet in the patient's trachea or windpipe and thereby prevent insertion into the patient's lungs. Typical weights for the base magnet range from about 3 to about 6 pounds, preferably from about 3 to about 5.5 pounds, more preferably from about 4 to 5 pounds.

Magnets having the above-listed dimensions that are formed from Neodymium typically have magnetic fields measured at 3 inches from the magnet ranging from about 500 to 600 Gauss. For example, Neodymium magnets having a diameter of 4 inches and thickness of 1.8 inches typically have a magnetic field of about 500 Gauss, when measured at 3 inches from the magnet.

The magnetic field of the external magnet combines with the magnetic field of the internal magnet to produce a sufficient magnetic force to pull or push the stylet against the posterior wall of the esophagus to prevent placement of the stylet in the trachea.

D. Dimensions for Magnet

The dimensions and material for the external magnet(s) are selected to provide a magnetic field that combines with the magnetic field of the internal magnet to produce a sufficient magnetic force to pull or push the stylet against the posterior wall of the esophagus to prevent placement of the stylet in the trachea. Typical magnetic fields for the external magnetic range from 50 Gauss to 1,000 Gauss, preferably from 200 Gauss to 1,000 Gauss, more preferably 250 to 600 Gauss, and most preferably 400 to 600 Gauss, when measured at a distance 3 inches from the external magnet.

The external magnet (2) can have any suitable shape or size that allows it to be placed on the back or the front of a patient's neck. As shown in FIG. 1A, the magnet may be in the shape of a rectangle. However, other shapes may be used.

In another embodiment, the face of the magnet that is in contact with the patient's body is in the shape of an oval (egg shape) with a “double hull” shape (see FIG. 1B). This shape of the external magnet is selected to facilitate moving the external magnet along the spine posteriorly. This shape allows for the magnet to slide on the spine.

In one embodiment, the magnet is formed from a flexible material that can be bent to fit along the back of the patient's neck. In another embodiment, the magnet may be rigid.

In one embodiment, more than one magnet is used. Preferably, in this embodiment, the neck brace or collar contains the same number of pocket as the number of magnets. In one embodiment, three magnets (2a, b and c) are inserted into the neck brace. In another embodiment, four magnets (2a, b, c, and d) are inserted into the neck brace. Preferably the magnets are of the same size and shape. However, one or more of the magnets may have a size and/or shape that is different from the other magnets. In the preferred embodiment, the magnets are all rectangles (see FIGS. 1C and 1D).

Typical thicknesses for the magnets range from ⅛ inch to ¾ inch, preferably the magnets are approximately 1.4 inch thick. Typical widths for the magnets range from 2 inches to 6 inches. Typical heights for the magnets range from 1 inch to 4 inches. While these dimensions are particularly useful for magnets in the shape of a rectangle, the dimensions can be modified to accommodate different shaped magnets. Typically, for magnets having the same range of thicknesses listed above, the remaining dimensions for non-rectangular shaped magnets are selected to provide a magnet with the same surface area as a magnet in the shape of a rectangle having the above-referenced dimensions.

The preferred material for the base magnet is Neodymium N50 grade, which can be used to form a small and light weight magnet that provides the highest practical magnetic flux for its size and weight. A neodymium magnet (also known as NdFeB, NIB, or Neo magnet), a type of rare-earth magnet, is a permanent magnet made from an alloy of neodymium, iron, and boron to form the Nd₂Fe₁₄B tetragonal crystalline structure. This material is currently the strongest type of permanent magnet.

In one embodiment, the external magnet is an electromagnet, instead of a permanent magnet.

i. Electromagnet

In one embodiment, the external magnet is an electromagnet instead of a permanent magnet. In this embodiment, the electromagnet is preferably attached to a modular power supply via a tether. The tether, in addition to carrying the conductors, may provide conduits for water circulation to cool the electromagnetic probe and leads. For safety purposes, the tether preferably has a braided ground wire. Thus if the conductive leads carrying the high current to drive the electromagnet were in any way exposed, the tether would short to ground, and the power supply would be immediately shut down.

Benefits to using an electromagnet in the external magnet in place of a permanent magnet include, allowing the magnetic field to be further localized (smaller probe focuses the energy better), resulting in a more focused and stronger magnetic field. This facilitates guiding the feeding tube to its desired location. Additionally, an external magnet that contains an electromagnet can be much lighter than an external magnet containing a permanent magnet, such as having a weight from about 1 to 3 pounds, to develop the same magnetic field. Further, the electromagnet is switch able, so that it can be easily turned off when not it is in use. This prevents accidental movement of the feeding tube apparatus out of its desired location due to movement of the external magnet.

Another benefit of an electromagnet is that it may eliminate the need for the reed switch on the stylet, further reducing cost and complexity of the stylet construction. This is possible since an electromagnet can monitor and indicate the presence of another ferrous material that is being attracted to it. As such, a numeric or graphical indicator on the electromagnet power supply could also serve as an indicator of whether there is a sufficient magnetic force between the internal and external magnets. In this embodiment, a reed switch is not needed in the stylet to interact with the indicator in the electromagnetic probe.

ii. Indicator

The external magnet may be used with an indicator, which emits a signal. The signal indicates that the magnetic force between the feeding tube catheter and the external permanent magnet is strong enough to use the external magnet to direct the feeding tube catheter to the posterior wall of the patient's esophagus. In one embodiment, the external magnet (2) contains an indicator. In another embodiment, the feeding tube contains an indicator (see section II(A) (d) below).

Any suitable signal may be used. In one embodiment the signal is a lit light emitting diode (LED). In another embodiment, the signal is a sound, such as a tone or buzzer. The signal indicates that the magnetic force between the feeding tube catheter and the external permanent magnet is strong enough to use the external magnet to direct the feeding tube catheter to the posterior wall of the patient's esophagus.

In a preferred embodiment, neither the catheter nor stylet contains an indicator. This embodiment is particularly useful if the tube is used as a gastric tube since gastric placement generally does not require indication to ensure or confirm accurate placement of the tube once the tube has traveled past the trachea. Further, tracheal avoidance can generally be achieved without the need for indication of magnet capture between the external magnet and the magnet or magnet stack in the feeding tube catheter (i.e. that the magnetic field of the external magnet combines with the magnetic field of the internal magnet to produce a sufficient magnetic force to pull or push the stylet against the posterior wall of the esophagus to prevent placement of the stylet in the trachea).

### II. Uses for the External Magnet

The external magnet may be used to guide any feeding tube apparatus that contains a magnet or magnet stack (referred to as an “internal magnet”) toward the back of a patient's esophagus. In one embodiment, a system containing the external magnet, as described herein, and a feeding tube apparatus that contains one or more internal magnets, as described herein, is provided for directing the feeding tube apparatus to the back of a patient's esophagus to prevent insertion into the patient's trachea. The system may contain additional optional components, such as a second external magnet, as described below.

The feeding tube apparatus includes, at least, a catheter (20) and a magnet or magnet stack. In one embodiment the magnet or magnet stack is affixed to the catheter. In another embodiment, the magnet or magnet stack is removable from the catheter. Typically the magnet or magnet stack is located at the distal end (24) of the catheter.

In one embodiment, the catheter contains a magnet or magnet stack permanently affixed within the catheter. Examples of this embodiment are described in U.S. Pat. No. 5,431,640 to Gabriel and U.S. Pat. No. 6,126,647 to Posey, et al., the disclosures of which are herein incorporated by reference.

In another embodiment, the catheter contains a removable magnet or magnetically attractive material, preferably located on a removable stylet. In one embodiment, the stylet contains more than one magnetic material (e.g. magnet or magnetically attractive material) referred to herein as a “magnet stack”. Examples of a stylet that contains a magnetic stack are described in U.S. Publication No. 2009/062772, the disclosure of which is incorporated herein by reference. Some of the embodiments of the feeding tube apparatus described in U.S. Publication No. 2009/062772 are also described below.

A. Feeding Tube Apparatus

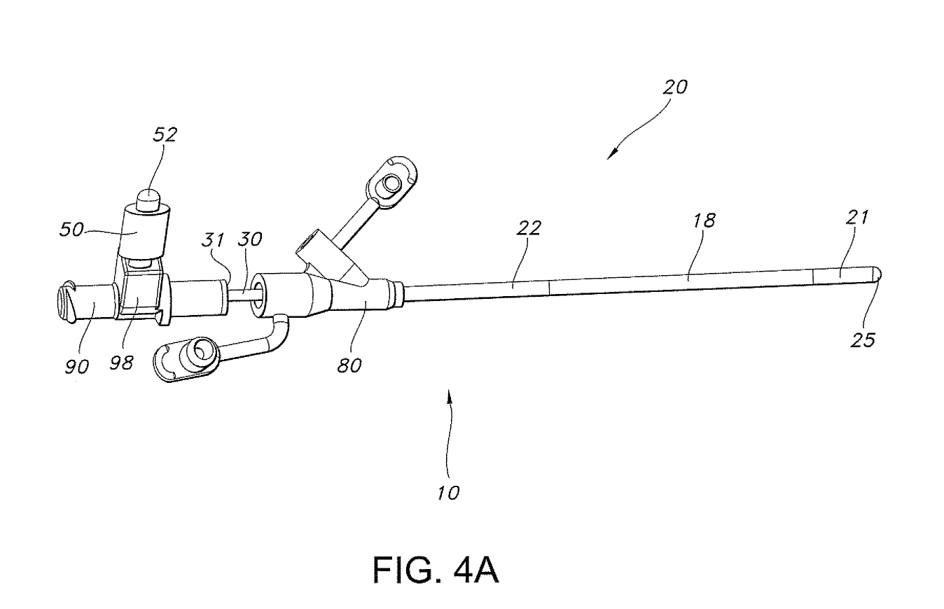

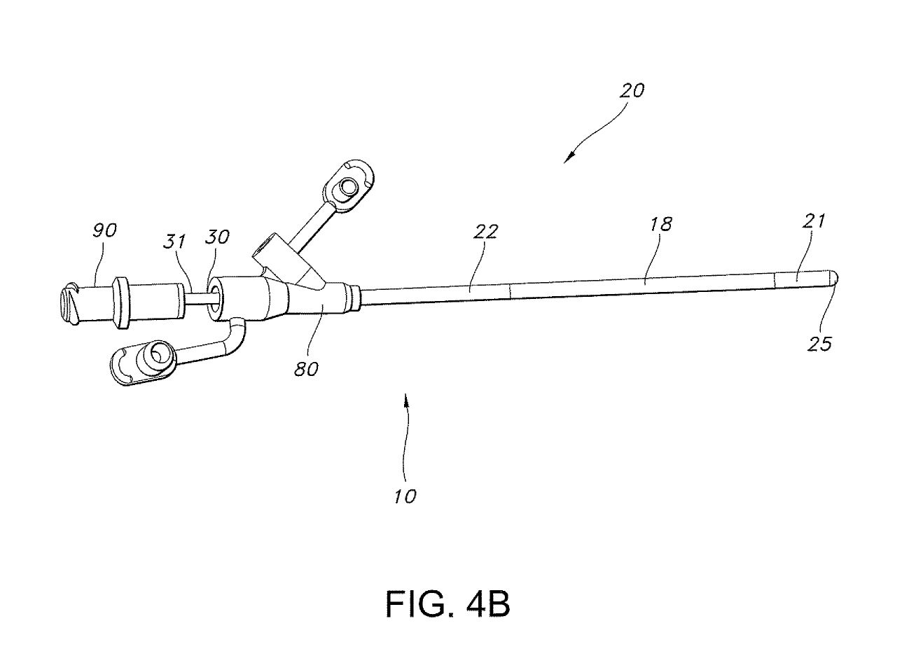

Representative feeding tube apparatuses are depicted in FIGS. 4A and B. The feeding tube apparatus includes a catheter (20) and a magnet or magnet stack. The catheter (20) is a tube with a proximal end (22) and a distal end (24). The distal tip (25) of the distal end (24) forms an open lumen (26). This allows for the delivery of food from the opening at the distal tip (25) of the catheter.

When the feeding tube apparatus is completely assembled, the catheter (20) is connected at its proximal end (22) to a feeding tube hub (80) and the stylet (30) is connected at its proximal end (31) to a stylet hub (90). The distal end of the stylet hub (90) is insertable into the feeding tube hub (80) and is removable therefrom. The distal end of the stylet hub (90) fits snugly in the feeding tube hub (80).

The open lumen (26) at the distal tip (25) also allows for the use of a fiberscope, i.e. a flexible, small endoscope, or other suitable device that can be placed through the open lumen at the distal tip to verify the location of the catheter. The use of a fiberscope can eliminate the need for X-rays to be taken to verify the location of the catheter.

a. Materials

The catheter may be formed of any suitable tubing. Typical tubing materials have a flex modulus ranging from about 500 psi to about 50,000 psi, preferably from 700 psi to 3,000 psi, most preferably about 1,500 psi.

The catheter may be formed of one material. In one embodiment, the catheter is formed from a flexible material, such as polyurethane or silicon tubing, with a flex modulus ranging from about 500 psi to about 30,000 psi.

In one embodiment the catheter is formed of two or more materials. For example, the distal tip (25) and the region proximal to the distal tip (21) are preferably formed of a softer material than the material that forms the rest of the catheter (20). This allows the distal tip (25) and the region proximal to the distal tip (21) to be atraumatic and allows the magnets to have a more pronounced effect on maneuverability and guidance than they would if a stiffer material was used.

For example, the tubing that forms the catheter may be dual durometer tubing, with at least two levels of flexibility; where the flex modulus for a first, softer portion is lower than the flex modulus for a second, more rigid portion. In one embodiment, the proximal end (22) comprises a first, relatively soft material, and the distal end (24) is more rigid than the proximal end. Preferably the tubing is relatively soft at the catheter's proximal end (22), at its distal tip (25) and at the region (21) proximal to the distal tip (25), which generally corresponds with the location of the magnetic material(s), and is more stiff in the region (18) between the proximal end (22) and the region (21) proximal to the distal tip (25). The soft material at the proximal end (22), which will be in contact with the patient's throat and nose, causes less irritation to the patient than a stiffer material. The soft portion of the catheter typically has a flex modulus ranging from about 500 psi to 30,000 psi, preferably ranging from about 750 psi to 3,000 psi. The stiffer material in the region (18) between the proximal end (22) and the region (21) proximal to the distal tip (25) allows the catheter to have greater pushability and maneuverability during insertion than if a softer material was included in this region (18) of the catheter. The stiffer portion of the catheter typically has a flex modulus ranging from about 1,500 psi to about 100,000 psi, preferably from about 10,000 psi to about 50,000 psi. The soft material at the distal tip (25) and at the region (21) proximal to the distal tip (25) allows the catheter to be atraumatic and allows the magnetic material(s) to have a more pronounced effect on maneuverability and guidance of the feeding tube apparatus.

Optionally, the catheter is constructed in whole or in part of a radiopaque material. Suitable materials include polyurethane or silicon tubing. The tubing is preferably comprised of a polyurethane for strength. Preferably the polyurethane does not soften or change significantly at body temperature. Examples of suitable polyurethanes include those available under the tradenames ESTANE® (Lubrizol Advanced Materials, Inc.), PEBAX® (Arkema France Corp.), PELLETHANE® (Dow Chemical Co.), and CARBOTHANE® (Lubrizol Advanced Materials, Inc.).

Preferably the outside of the catheter contains markings, which indicate the length of the catheter, and, in use, the length of the portion of the catheter that is inserted into the patient's body.

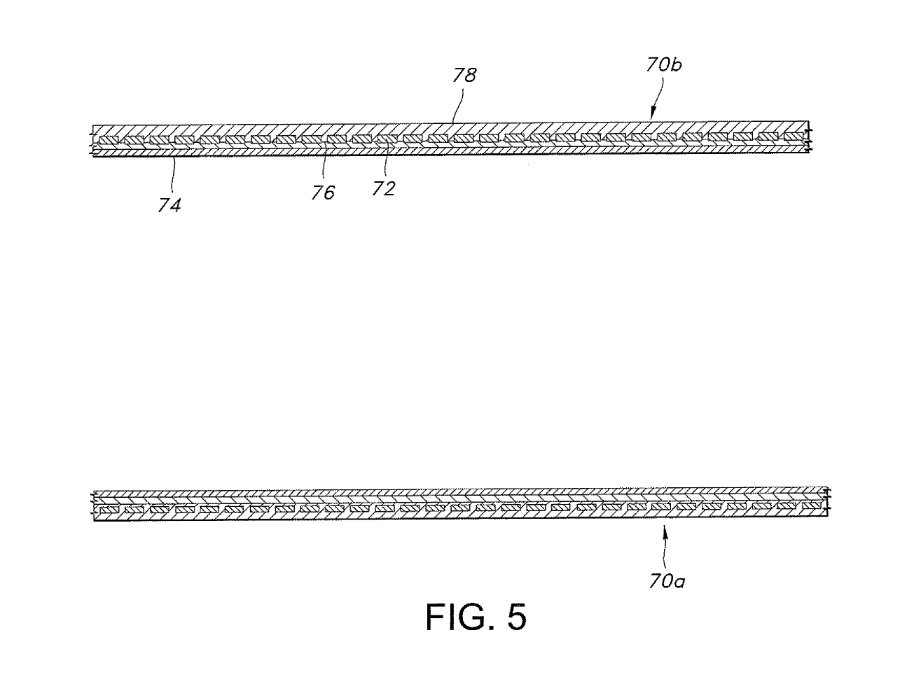

In another embodiment, the walls of the catheter contain a reinforcing material (see FIG. 5). In this embodiment, the walls (70a and 70b) of the catheter contain an MRI compatible reinforcing material (72), such as a fiber, monofilament, or non-ferrous metal. This allows the catheter to have a thin wall, while maintaining the desired inner diameter. A reinforcing material also provides kinking- and/or crush-resistance to the catheter. A reinforcing material also allows the catheter to be especially resilient to perforation, thereby facilitating the use of a plunger to purge a clogged catheter without the risk of perforating or damaging the feeding tube, even when the tube is conforming to a tortuous path in the patient's body. In addition, the reinforced construct also allows for reduced internal friction to facilitate stylet removal. Thus comparing a catheter without the reinforcing material with one containing the reinforcing material, the two catheters can have the same inner diameter, but the catheter with the reinforcing material can have a smaller outer diameter than the catheter without a reinforcing material while maintaining the same wall strength. Typical thicknesses for the walls of a catheter without a reinforcing material range from 0.020 inches to 0.025 inches. For catheters with a reinforcing material, typical thicknesses for the walls of the catheter range from 0.008 inches to 0.020 inches. This difference between the wall thicknesses results in a catheter with a reinforcing material with an outer diameter that is one to two French sizes smaller than the outer diameter for a catheter without a reinforcing material, while maintaining the same inner diameter and the same wall strength.

Suitable reinforcing materials are stiff, MRI compatible, i.e. non-ferrous, materials, such as, polyester, copper, aluminum, non-magnetic stainless steel, or other non-ferrous, stiff, materials. In the preferred embodiment, the reinforcing material is polyester monofilament. However, the reinforcing material can be any stiff, non-ferromagnetic material, such as copper or other monofilament materials.

In one preferred embodiment illustrated in FIG. 5, the wall (70) of the catheter (30) contains a smooth inner layer (74), a binder layer (76), a reinforcing material (72), and an outer layer (78). The smooth inner layer (74) is typically formed of a material with a low coefficient of friction, i.e. about 0.3 or less, with typical ranges from about 0.1 to 0.4, preferably from 0.1 to 0.2. Suitable materials for the inner layer (74) include FEP (Fluorinated Ethylene Propylene copolymer), PFA (Perfluoroalkoxy), and PTFE (Polytetrafluoroethylene) TEFLON® (E. I. Du Pont de Nemours and Co.). The binder layer (76) is a soft and tacky layer that binds the inner layer (74) with the reinforcing material (72) and, optionally, the outer layer (78). Typical materials have a coefficient of friction, ranging from 0.6 to 1.5, preferably 1.0. A suitable material for the binder layer (76) is 25 D PEBAX®. The material for the outer layer (78) is selected based on the desired stiffness for the catheter, typical materials have a flex modulus ranging from about 500 psi to about 50,000 psi, preferably from 700 psi to 3,000 psi, most preferably about 1,500 psi. Suitable materials for the outer layer (78) include 35D, 40D, or 55D PEBAX®, 75A, 85A, 95A, or 55D CARBOTHANE®, or 80A, 85A, 90A, or 55D PELLETHANE®.

A catheter containing a reinforcing material (72) can be formed using any suitable method. In one embodiment, the catheter is formed by coating the inner layer (74) over a mandrel forming a tube. Then the binder layer (76) is coated over the inner layer (74) forming a coated tube. Next the reinforcing material (72) is spiral wrapped around the coated tube. This is preferably a continuous process. Then an outer layer (78) having the desired stiffness, is pressure-extruded or vacuum-assisted over extruded over the wrapped and coated tube. Then, the mandrel is removed. The resulting catheter (30) contains a reinforcing material (72) in the wall (70) of the catheter. This process produces a thin-walled, kink- and/or crush-resistant catheter.

Alternatively, the reinforced tube can simply contain its reinforcing member (72) on the inner surface and an outer layer (78). Suitable materials for the outer layer (78) include 35D, 40D, or 55D PEBAX®, 75A, 85A, 95A, or 55D CARBOTHANE®, or 80A, 85A, 90A, or 55D PELLETHANE®.

In another embodiment, the reinforcing material (72) is spiral wrapped around the mandrel forming a tube. This is preferably a continuous process. Then an outer layer (78) having the desired stiffness, is pressure-extruded or vacuum-assisted over extruded over the wrapped and coated tube. Then, the mandrel is removed. The resulting catheter (30) contains a reinforcing material (72) in the wall (70) of the catheter. This process produces a thin-walled, kink- and/or crush-resistant catheter.

b. Dimensions

Any standard diameter and length of tubing material may be used to form the catheter. The current standard catheter sizes are referred to as “French” sizes, e.g. size F4 refers to a tube with a 0.053 inch outer diameter, F5 refers to a tube with a 0.066 inch outer diameter, F6 refers to a tube with a 0.079 inch outer diameter, F7 refers to a tube with a 0.092 inch outer diameter, F8 refers to a tube with a 0.104 inch outer diameter, F10 refers to a tube with a 0.103 inch outer diameter, F11 refers to a tube with a 0.143 inch outer diameter, and F12 refers to a tube with a 0.156 inch outer diameter. In a preferred embodiment, the tubing is a single lumen 2603-80AE PELLETHANE® F11 or F12 tube. The F11 tube has an outer diameter of 0.143 inches and an inner diameter of 0.111 inches; and the F12 tube has an outer diameter of 0.156 inches and an inner diameter of 0.116 inches. However other size tubing is suitable as well. In place of single lumen tubing, double lumen tubing or alternative styles may be used. The inner diameter of the tubing (i.e. the diameter of the lumen) should be sufficiently large to allow the fluids and nutrients to pass through the catheter without clogging the catheter. Preferably the inner diameter of the tubing (i.e. the diameter of the lumen) is sufficiently large to allow particles with a diameter of up to 0.110 inches to pass through the tubing.

The length of the catheter determines how deep into the gut the feeding tube can be placed for the delivery of fluids and nutrients. Typical lengths for the catheter range from 100 cm to 150 cm. In one embodiment, the catheter ranges from 100 to 125 cm long. This allows for gastric placement of the feeding tube apparatus. In another embodiment, the catheter is at least 125 cm long, and preferably is 127 cm long. This allows for the nutrients to be delivered deep into the bowel and thereby prevent reflux. Catheters that are at least 100 cm long prevent the patient from inadvertently removing the feeding tube after placement in the stomach such as through standard movements.

c. Stylet with Removable Magnet or Magnet Stack

Optionally, the catheter contains a removable stylet (30), where the removable stylet contains one or more magnets (32a-e). Removing the stylet subsequent to successful catheter placement makes the assembly “MRI safe”, i.e. allows for a patient to be imaged using MRI. Flexibility is required for the distal end of the stylet to facilitate movement of the stylet through the patient's body. Further, the stylet must have sufficient lubricity to be pulled out of the catheter, particularly when the catheter is in place and has conformed to the various curves in the patient's body.

i. Internal Magnet or Magnet Stack

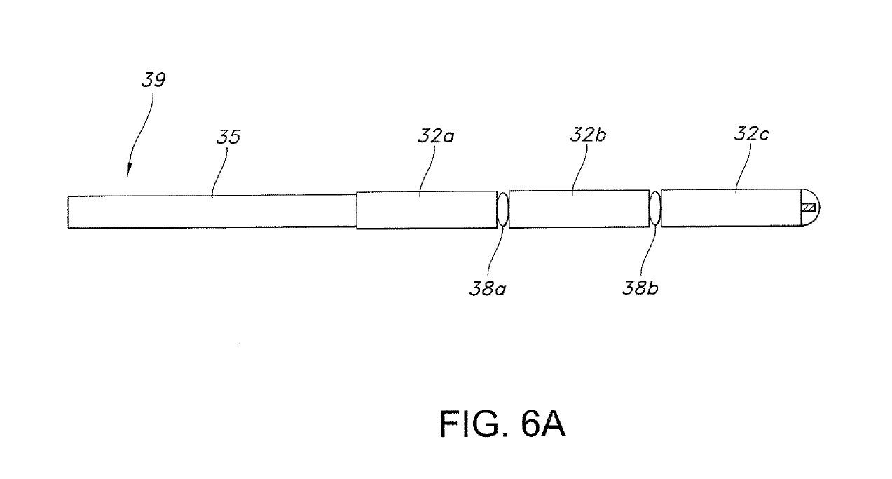

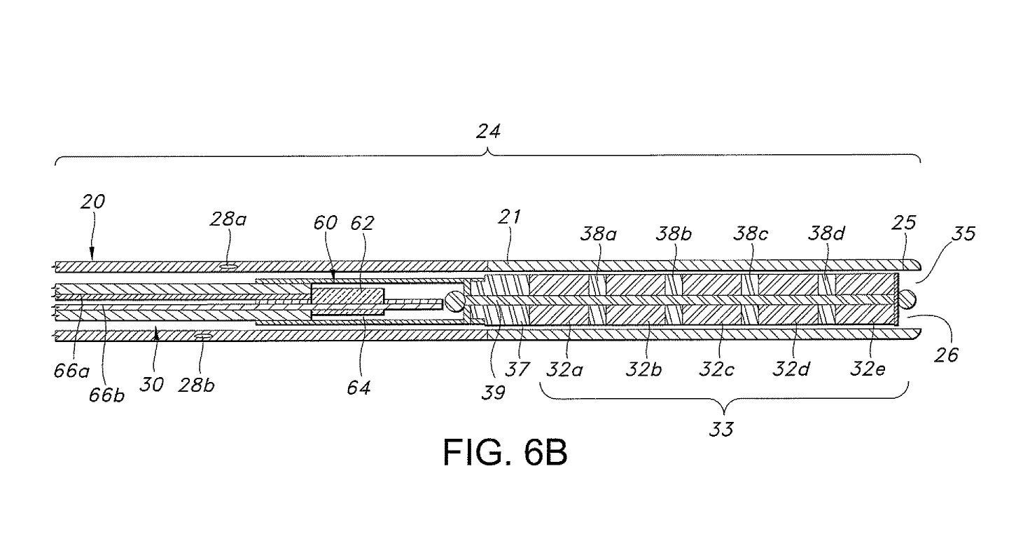

In a preferred embodiment, the distal end (34) of the stylet (30) contains one or more magnets (32) (the “internal magnet”), and preferably contains a plurality of magnets, referred to herein as a “magnet stack” (33). The length of the magnet or magnet stack can be any suitable length for obtaining the necessary magnetic force between the external magnet (2) and the internal magnet or magnet stack in the stylet. Typical lengths for the internal magnet (32) range from 0.01 inches to 0.200 inches, preferably from 0.100 to 0.200 inches. Typical lengths for the magnet stack (33) range from 0.25 inches to 2.0 inches, preferably the magnet stack (33) is about 0.75 inches long. In a preferred embodiment, the magnet stack (33) contains two or more stacked magnets with each magnet having the same dimensions. Preferably the magnets are connected to each other by a connecting element, such as a core wire (39) adhesive, coating, or a heat shrink tube, such as polyester shrink, or combination thereof. In a preferred embodiment, the wire (39) extends through a hole in the center of each magnet (see FIGS. 6A and B). The connecting element is preferably suitably affixed to the magnet stack to ensure that it remains connected with the magnet stack. For example, the wire (39) may terminate at the distal end of the magnet stack (33) with a material that is wider than the hole in the magnet stack, such as in the form of a bead.

In a preferred embodiment, the magnet stack (33) contains three stacked magnets (32a-c) with each magnet having a length from 0.35 to 0.45 inches, preferably the length of each magnet is 0.400 inches (see FIG. 6A). In one embodiment, the magnet stack (33) contains five stacked magnets (32a-e) with each magnet having a length from 0.1 inches to 0.125 inches, preferably the length of each magnet is 0.125 inches (see FIG. 6B). For additional flexibility each magnet in the magnet stack may be separated from the proximal magnet with a flexible spacer (38a-d), such as an O-ring, or a spacer formed from a soft polyurethane or silicon. This facilitates movement of the magnet stack through the patient's body, particularly along curves when the catheter and stylet are conforming to a tortuous path in the patient's body.

The diameter of the magnet(s) is selected based on the inner diameter of the catheter. The outer diameter of the magnet(s) is less than the inner diameter of the catheter so that the magnet(s) can easily slide into and out of the catheter, as desired. In a preferred embodiment, the diameter of the magnet or magnet stack is greater than the diameter of the stylet, and less than the inner diameter of the catheter. This allows the magnet or magnet stack to provide the greatest magnetic force for the area within the catheter, and seals the opening at the distal tip (26) when the stylet is inside the catheter. By sealing the distal tip, when aspiration occurs, it will generally only occur through the sideholes (28a and 28b), and will not also occur at the distal tip (26). By way of example, for catheters formed using 11 FR or 12 FR tubing, the magnet(s) may have an outer diameter 0.050 inches to 0.125 inches, with a preferred diameter of 0.105 inches.

ii. Core Wire

In a preferred embodiment, the stylet is formed essentially by a core wire (39) where the wire runs the length of the stylet. In one embodiment, the core wire passes through the magnet or magnet stack. In another embodiment, the core wire merely attached through a suitable attachment means, such as an adhesive, or heat shrink tube, such as polyester shrink, or a combination thereof, to the magnet or proximal magnet in a magnet stack. Exemplary materials for the wire (39) include nylon coated stainless steel stranded wire, such as a 7×7 wire or a 7×19 wire (Fort Wayne Metals, California Fine Wire Co., New England Wire Technologies Corp., or Delco Wire Winding Co., Loos & Co., Inc.). A stranded wire provides good tensile strength as well as good flexibility for maneuvering the catheter through the patient's body. The proximal portion of the stylet (31) up to the most proximal magnet (32a) is preferably stiffened by suitable means, such as over extruding on the nylon coated stranded wire, a suitable material (35), which is selected based on the overall flex modulus required and allows sufficient lubricity to facilitate removal of the stylet from the catheter. Suitable coating materials include but are not limited to nylon 6/6, polyurethane, polyolefin, PVC. For a flex modulus of about 10,000 psi, typical materials include nylon 6/6 and polyolefin. The final diameter of the overextrusion coated stranded wire is selected so that the stylet fits inside the catheter and is removable therefrom once the catheter is the desired location within the patient's body; suitable overall diameters range from 0.019 inches to 0.085 inches.

d. Reed Switch Assembly

In one embodiment, the catheter or the removable stylet (30), contains a reed switch assembly (36) to ensure that an indicator (52) properly indicates when the magnetic force between the magnet(s) (32) or magnetic stack (33) and the external magnet (40) is sufficiently strong for the external magnet (40) to guide the catheter (20) along the intestinal tract. However, a reed switch assembly is not required for a feeding tube apparatus that does not include an indicator, such as those used for gastric placement of the feeding tube.

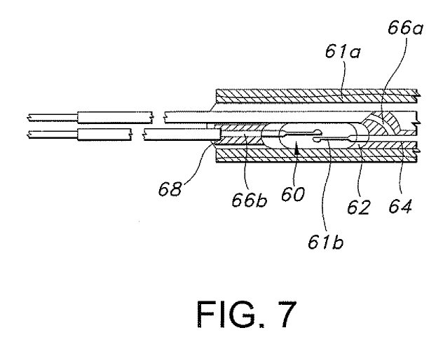

An example of a suitable reed switch assembly (36) is described in U.S. Pat. No. 6,126,647 to Posey and is illustrated in FIGS. 6 and 7.

The reed switch assembly (36) contains reeds (60a and 60b) sealed in a glass envelope (62), which is disposed within a metal housing (64). The leads (66a and 66b) are soldered to the external portions of the reeds (60a and 60b), and the metal housing (64) of the reed switch assembly (36) is affixed to the stylet (30) and thereby connects the reed switch assembly (36) the spacer (37), the magnet (32) or magnet stack (33), and, optionally the spring wire guide. In a preferred embodiment, the magnet (32) or magnet stack (33) is coated with a biocompatible coating that also provide a lubricious surface, such as parylene, to facilitate easy sliding of the stylet in and out of the feeding tube. Alternatively, a heat shrink tube, such as polyester shrink, can also be employed to encapsulate this assembly and thereby ensure that the reed switch assembly (36) the spacer (37), the magnet (32) or magnet stack (33), and, optionally the spring wire guide, do not separate from the stylet (30). Heat shrink tubing also provides a lubricious surface between the inner surface of the catheter and the stylet (30) to facilitate insertion and removal of the stylet (30) from the catheter (20).

For an external magnet (40) having a magnetic field ranging from 100 Gauss to 1,000 Gauss, and preferably from 200 Gauss to 1,000 Gauss, and more preferably from 250 to 600 Gauss, and most preferably from 400 to 600 Gauss, at a distance of 3 inches from the external magnet (40), the reeds (60a and 60b) in the reed switch assembly (36) will contact each other, thereby actuating the indicator (52), when the external magnet (40) is within 3 to 5 inches of the reed switch (36). In one preferred embodiment, the reeds contact each other, thereby actuating the indicator (52), when the external magnet (40) is within about 3 inches of the reed switch assembly (36).

The indicator (52) produces a signal when it is actuated. Any suitable signal may be used. In one embodiment the signal is a lit LED. In another embodiment, the signal is a sound, such as a tone or buzzer. The signal indicates that the magnetic force between the feeding tube catheter and the external permanent magnet is strong enough to use the external magnet to direct the feeding tube catheter to the back of the patient's esophagus.

The dimensions of the spacer (37) are selected based on the distance between external magnet (40) and the reed switch (36) required to actuate the indicator (52). For example, in one embodiment, spacer (37) between the reed switch (36) and the proximal end of the magnet (32) or magnet stack (33) is at least 2 mm long. A feeding tube apparatus (10) designed in this manner will typically produce a signal when the external magnet (40) is within about 4 inches of the reed switch (36).

Although the stylet (30) is illustrated herein as containing a normally open reed switch assembly (36) disposed within a metal housing (64), other reed switches, such as those that are normally closed, may be used. A single-pole, single-throw (SPST), normally-open reed switch (also referred to as a Form “A” reed switch) is illustrated herein. Single-pole, single-throw (SPST), normally-closed reed switches (also referred to as Form “B” reed switches), single-pole, double-throw (SPDT), and break-before-make reed switches (also referred as Form “C” reed switches) are known in the art and may be used in place of the reed switch (36) depicted in FIGS. 6 and 7. Regardless of the particular type of switch used, e.g. Form A, Form B, or Form C, each type of reed switch can be used as described herein.

e. Sideholes

In addition to the openings at the proximal and distal ends of the catheter, the catheter optionally contains one or more holes along the wall of the catheter, referred to herein as “sideholes” (28), in the region proximal to the region proximal (21) to the distal tip (25) of the catheter. The sideholes ensure that, even if the feeding tube is lodged against a wall in a patient's body, aspirating the catheter will not create a suction situation and potentially damage internal tissues or walls.

Preferably the sideholes are located as close to the distal tip (25) as possible without compromising the strength of the tubing and interfering with the magnet, (32) or magnet stack (33) and reed switch assembly (36). In one embodiment, the catheter contains two sideholes (28a and 28b). The sideholes are typically oval or circular in shape and typically have dimensions ranging from 0.010 inches to 0.110 inches; preferably the sideholes have diameters of about 0.050 inches.

In one embodiment, the distal end of the catheter does not contain an opening, but the catheter contains one or more sideholes. In this embodiment, the nutrients are delivered through the sideholes.

B. Optional Components

Optionally the external magnet (2) and the feeding tube apparatus (10) are used with one or more additional components. Suitable additional components include a syringe, preferably a 60 CC syringe; one or more towels; one or more cups, preferably at least two cups; disposable gloves; Xylocaine gel (e.g. 2% Xylocaine gel); tape; gauze; disposable magnet covers; spring wire guide, and/or pH paper. Optionally, the feeding tube apparatus (10) contains a plunger that can clear clogs in the catheter to eliminate the need to remove the catheter and replace it with another one.

In one preferred embodiment, the feeding tube apparatus (10) contains a stylet that is insertable into and removable from the catheter (herein the “removable stylet”).

Optionally, the feeding tube apparatus (10) contains a spring wire that is insertable into and removable from the catheter. In one embodiment, a kit containing the feeding tube apparatus also includes a removable stylet and a spring wire guide.

a Second External Magnet

In some embodiments, the external magnet may also be used as a guide to direct the feeding tube apparatus through the stomach, such as for post pyloric placement of the feeding tube apparatus. In these embodiments, a handheld external magnet is particularly useful (see e.g. FIGS. 3A-C).

In other embodiments, particularly when the magnet is in a form suitable for placement in a neck brace or collar, a second external magnet may be used as a guide to direct the feeding tube apparatus through the stomach. In these embodiments, the second external magnet is typically heavier and larger than the first external magnet.

The second external magnet (40) can have any suitable shape or size that allows manipulation by the healthcare provider. FIGS. 3A-C depict three representative designs for the external magnet. As noted above with respect to the first external magnet, the second external magnet may be formed from a permanent magnet or an electromagnet.

In one embodiment, a second external magnet is used solely to identify or verify the location of the distal tip of the feeding tube apparatus within a patient's body following guidance with the first external magnet. In this embodiment, the second external magnet is not designed to guide the feeding tube through the patient's body. In this embodiment, the second external magnet is typically smaller and preferably lighter than the first external magnet. The second external magnet has a magnetic field that is less than the magnetic field needed to interact with the magnetic field of the internal magnet to create a magnetic force sufficient to guide the internal magnet (either by pushing or pulling) through the patient's body. In one embodiment, such as for a feeding tube apparatus that contains a reed switch, the magnetic field of the second external magnet is sufficient to close the reed switch and thereby produce a signal when the external magnet is at distance of 3 inches or less from the reed switch. The second external magnet has a sufficient magnetic field to close the reed switch circuit when the magnetic field is measured at a distance of about three inches or less, or a distance of about 2 inches or less from the second external magnet and the second external magnet has an insufficient magnetic field to close the reed switch circuit when the magnetic field is measured at a distance of about five inches or more from the second external magnet. For example, in one embodiment, the second external magnet has a magnetic field of about 200 Gauss or more, when measured 3 inches from the external magnet or when measured 2 inches from the magnet, and less than 200 Gauss, when measured at 5 inches from the magnet. In contrast, the first external magnet, which is used to guide the feeding tube, typically has a magnetic field of about 200 Gauss or more when measured at a distance of 5 inches from the external magnet. The reduction in magnetic field (and 2 inch distance) seen with the second external magnet translates into greater location accuracy and placement confidence.

In one embodiment, the second external magnet contains an indicator, that indicates when the reed switches connect.

b. External Sensor

Optionally, an external sensor may be used to identify the location of the distal tip of the feeding tube apparatus within the patient's body. The external sensor contains a means with a sufficient accuracy to sense the presence of the internal magnets within a distance of five inches or less from the internal magnet(s). Suitable means include a gaussmeter and other comparable magnetic field measuring sensors. The external sensor may also include an indicator, such as an LED, or other suitable indicator, to identify to a user that the external sensor sensed the presence of the one or more internal magnets and thereby identify the location of the distal tip of the feeding tube apparatus.

### III. Method of Using the External Magnet

The relative polarities of the internal and external magnets determines whether the external magnet is placed on the front of the patient's neck or the back of the patient's neck.

If the external magnet has the same polarity as the internal magnet, then the external magnet is placed on the front of the patient's neck to guide the feeding tube by pushing (due to the repellant force between the internal and external magnets) the feeding tube to the posterior wall of the esophagus, thereby avoiding insertion of the feeding tube into the trachea.

If the external magnet has the opposite polarity as the internal magnet, then the external magnet is placed on the back of the patient's neck to guide the feeding tube by pulling (due to the attractive force between the internal and external magnets) the feeding tube to the posterior wall of the esophagus, thereby avoiding insertion of the feeding tube into the trachea.

In a preferred embodiment, the patient is placed on his/her side to facilitate insertion and guidance of the feeding tube apparatus. Alternatively, the patient may be in a sitting position.

First the external magnet is applied to either the front or the back of the patient's neck, depending on the relative polarities of the internal and external magnets. In one embodiment, the external magnet is placed inside a device, such as a neck brace or collar, that is affixed to the patient's neck. In another embodiment, the external magnet is placed in or on a pillow that is placed behind a patient's neck. In a preferred embodiment, the external magnet is a handheld magnet, with a base magnet, preferably in the shape of a cylinder, and a handle, where the base magnet is about 4 inches in diameter and about 2 inches in height.

Then, the distal tip (25) of the catheter (20) is introduced into the naris of the nose and advanced by the continued application of a compressive force to the catheter forcing the distal tip (25) of the catheter (20) to the posterior wall of the patient's esophagus. A sufficient magnetic force between the external magnet and the magnet in the catheter ensures that the distal tip of the catheter is guided through the esophagus by directing the distal tip of the catheter to the posterior wall of the esophagus, thereby preventing the distal tip of the catheter from entering trachea.

In a preferred embodiment for the posterior advancement of the feeding tube the patient remains in lateral decubitus to allow the medical practitioner to access the esophagus posteriorly and follow the tube anteriorly when it reaches the stomach. This position also allows the tube to be guided easily into the duodenum.

The feeding tube is placed through the nasopharynx into the hypopharynx, and directed posteriorly through the upper esophageal sphincter (UES). The external magnet is preferably placed posteriorly on the cervical spine and then sweeped posteriorly following the thoracic spine into the lumbar spine.

In one embodiment, the feeding tube contains an indicator, such as an LED, that confirms to the medical practitioner that the feeding tube is following the midline (i.e. following the esophagus). If there is a loss of magnetic connection between the external magnet and the one or more internal magnets in the distal tip of the feeding tube, the indicator turns off (e.g. LED turns off) and thereby indicates to the medical practitioner that the feeding tube is misplaced to the right or the left into the bronchus. Thus, this technique of tube placement avoids placement of the feeding tube in the airway and provides early detection of misplacement, in the unlikely event this would occur.

In a preferred embodiment, the magnet is placed on the back of the patient's neck. Then, the distal tip (25) of the catheter (20) is introduced into the naris of the nose and advanced by the continued application of a compressive force to the catheter forcing the distal tip (25) of the catheter (20) to the posterior wall of the patient's esophagus. Preferably the outside of the catheter contains markings, which indicate the length of the catheter, and the length of the portion of the catheter that is inside the patient's body. After approximately 15 to 30 cm, preferably 20 to 30 cm, of the length of the catheter is inserted into the patient, a medical practitioner may move the external magnet down the patient's body along the patient's spine, while advancing the feeding tube inside of the patient, until the external magnet reaches the top of the lumbar region of the patient's back, i.e. the region of the back lateral to the vertebral region and located between the rib cage and the pelvis.

In one embodiment, the external magnet contains an indicator, which emits a signal when the magnetic force between the feeding tube catheter and the external permanent magnet is strong enough to use the external magnet to direct the feeding tube catheter to the posterior wall of the patient's esophagus. In this embodiment, when the indicator emits a signal, the medical practitioner, moves the external magnet down along a patient's spine, while continuing to advance the tube, until the external magnet reaches the lumbar region of the patient's back, as described above.

Although not required, the placement of the tube may be confirmed using any suitable method or device, including an indicator on the feeding tube apparatus or external magnet, CO₂ monitors, such as an end-tidal CO₂ (EtCO₂) monitoring systems; pH sensors, such as a continuous pH sensor; and endoscopes.

The external magnet may be removed from the patient's neck after the distal tip of the catheter descends within the patient's body past the neck. For example, if the external magnet is placed in a pillow, it is removed from the pillow. In some embodiments, the external magnet is then applied to the patient's abdomen to guide the placement of the feeding tube, such as for post pyloric placement of the feeding tube.

In some embodiments, a hand held external magnet (40), which may be the same as or different from the first external magnet, is applied to the patient's body within the minimum distance required to create a magnetic force between the external magnet (40) and the magnet (32) or magnet stack (33) that is sufficiently strong to allow the external magnet (40) to guide the feeding tube apparatus (10) through the stomach, the annular notch, the pyloric sphincter, into the duodenum portion, and into the distal duodenum of the small intestine.

In other embodiments, the medical practitioner continues to guide the catheter within the patient's stomach without the aid of an external magnet.

Optionally, the pH of the environment around the catheter distal end as the feeding tube apparatus is measured as the feeding tube apparatus is maneuvered through the patient to help determine when the feeding tube apparatus reaches the desired location for placement.

If a removable stylet is inside the catheter, the stylet is typically removed from the catheter when the distal tip of the catheter is placed in its desired location.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. An external magnet, wherein the external magnet has a size and shape suitable for placement on a patient's neck and comprises one or more magnets having a magnetic field selected to combine with the magnetic field of an internal magnet to produce a sufficient magnetic force to pull or push a feeding tube comprising the internal magnet against the posterior wall of a patient's esophagus to prevent placement of the feeding tube in the patient's trachea.

2. The external magnet of claim 1, wherein the magnet has a magnetic field ranging from 100 Gauss to 1,000 Gauss, when measured at a distance 3 inches from the magnet.

3. The external magnet of claim 2, wherein the magnetic field ranges from 400 to 600 Gauss, when measured at a distance 3 inches from the magnet.

4. The external magnet of claim 1, wherein the magnet is a permanent magnet.

5. The external magnet of claim 1, wherein the magnet is an electromagnet.

6. The external magnet of claim 1, wherein the magnet is located within a pillow, neck brace or cervical collar.

7. The external magnet of claim 6, wherein more than one magnet is located in the pillow, neck brace or cervical collar.

8. The external magnet of claim 6, wherein the pillow, neck brace or cervical collar comprises pockets suitable for placement of the magnet.

9. The external magnet of claim 1, further comprising an indicator.

10. A method for guiding a feeding tube through a patient's esophagus, comprising placing an external magnet on the back or the front of a patient's neck, and
inserting a feeding tube in the patient's naris, wherein the feeding tube comprises one or more internal magnets,
wherein the magnetic field of the external magnet is selected to combine with the magnetic field of the one or more internal magnets to produce a sufficient magnetic force to pull or push the feeding tube against the posterior wall of the esophagus to prevent placement of the feeding tube in the trachea.

11. The method of claim 10, wherein the external magnet has a magnetic field ranging from 100 Gauss to 1,000 Gauss, when measured at a distance 3 inches from the magnet.

12. The method of claim 11, wherein the external magnet has a magnetic field ranging from 400 to 600 Gauss, when measured at a distance 3 inches from the magnet.

13. The method of claim 10, wherein the external magnet comprises a permanent magnet.

14. The method of claim 10, wherein the external magnet comprises an electromagnet.

15. The method of claim 10, further comprising, after inserting approximately 15 to 30 cm of the length of the feeding tube into the patient, moving the external magnet down the patient's body along the patient's spine, and continuing to advance the feeding tube inside of the patient, until the external magnet reaches the top of the lumbar region of the patient's back.

16. The method of claim 10, wherein the feeding tube comprises a removable stylet, and wherein the one or more internal magnets are on the distal end of the removable stylet.

17. The method of claim 10, wherein the polarity of the one or more internal magnets is the opposite of the polarity of the external magnet, and wherein the external magnet is placed on the back of the patient's neck.

18. The method of claim 10, wherein the polarity of the one or more internal magnets is the same as the polarity of the external magnet, and wherein the external magnet is placed on the front of the patient's neck.

19. The method of claim 10, further comprising removing the external magnet after the feeding tube has descended past the patient's neck.

20. The method of claim 10, further comprising placing the same or a different external magnet on the patient's abdomen to guide the feeding tube through the patient's stomach, and
guiding the feeding tube through the patient's stomach using the magnetic field between the external magnet and the one or more internal magnets to the desired site.

21. The method of claim 10, further comprising placing a second external magnet on the patient to determine the location of the one or more internal magnets within the patient,
wherein the second external magnet has a magnetic field that is less than the magnetic field required to interact with the magnetic field of the one or more internal magnets to create a magnetic force sufficient to guide the feeding tube through the patient's body.

22. The method of claim 21, wherein the feeding tube apparatus comprises a reed switch, and wherein the magnetic field of the second external magnet is sufficient to close the reed switch circuit when the magnetic field is measured at a distance of about three inches or less, or a distance of about 2 inches or less from the second external magnet and the magnetic field of the second external magnet is insufficient to close the reed switch circuit when the magnetic field is measured at a distance of about five inches or more from the second external magnet.

23. A feeding tube guidance system comprising an external magnet with a size and shape suitable for placement on a patient's neck, and a feeding tube apparatus comprising one or more internal magnets,
wherein the magnetic field of the external magnet is selected to combine with the magnetic field of the one or more internal magnets to produce a sufficient magnetic force to pull or push the feeding tube apparatus against the posterior wall of a patient's esophagus to prevent placement of the feeding tube in the patient's trachea.

24. The feeding tube guidance system of claim 23, wherein the external magnet has a magnetic field ranging from 100 Gauss to 1,000 Gauss, when measured at a distance 3 inches from the magnet.

25. The feeding tube guidance system of claim 23, wherein the external magnet has a magnetic field ranging from 400 to 600 Gauss, when measured at a distance 3 inches from the magnet.

26. The feeding tube guidance system of claim 23, wherein the external magnet comprises a permanent magnet.

27. The feeding tube guidance system of claim 23, wherein the external magnet comprises an electromagnet.

28. The feeding tube guidance system of claim 23, wherein the feeding tube apparatus further comprises a removable stylet, and wherein the one or more internal magnets are on the distal end of the removable stylet.

29. The feeding tube guidance system of claim 28, wherein the removable stylet consists essentially of a core wire that runs the length of the stylet, a coating on the wire, and the one or more internal magnets at the distal end of the stylet.
